(19) United States
(12) Patent Application Publication
(10) Pub. No.: US 2025/0375318 A1
(43) Pub. Date: Dec. 11, 2025

(54) AURAL DEVELOPMENT SYSTEM AND METHOD

(71) Applicant: Sonura, LLC, Philadelphia, PA (US)

(72) Inventors: Gabriella Daltoso, Boise, ID (US); Sophie Ishiwari, Chicago, IL (US)

(21) Appl. No.: 19/229,485

(22) Filed: Jun. 5, 2025

Related U.S. Application Data
(60) Provisional application No. 63/657,670, filed on Jun. 7, 2024.

(51) Int. Cl. A61F 11/14; G06F 3/16; H04R 1/10; H04R 3/04

(52) U.S. Cl. CPC A61F 11/145; G06F 3/162; G06F 3/165; H04R 1/1083; H04R 3/04; H04R 2460/01

(57) ABSTRACT

An acoustic aural development system. A medical device is worn by an infant while the infant is in a neonatal intensive care unit (NICU) and is in communication with an acoustic processing station. The device and station protect the child's hearing by active acoustic filtering, while, at the same time, provides aural stimulation for development. Acoustic filtering is done at the acoustic processing station to reduce power and electronics required by the wearable medical device.

### CROSS-REFERENCE TO RELATED APPLICATIONS

This application claims the benefit of priority to U.S. Provisional Application No. 63/657,670, filed on Jun. 7, 2024, the entire disclosure of which are hereby incorporated by reference.

### BACKGROUND

This specification relates to medical devices and systems, and in particular to a medical device and system that provides active filtering hearing protection for an infant while also providing an aural development environment for the infant.

Approximately 500,000 births annually result in newborn admissions to Neonatal Intensive Care Units (NICUs) in the United States. Unfortunately, while providing life-saving treatments and therapies, NICUs expose newborns to high-frequency noises and levels for over twelve hours every day. At these levels and duration, such noises may potentially damage the newborns' hearing. In particular, monitors and alarms surrounding each crib emit a multitude of noises that may upset or startle the patients and contribute to a hazardous auditory environment for the patients. Such exposure has long-term consequences for “graduates,” i.e., infants who have been discharged from the NICU. These children face an increased risk of hearing impairment and potential learning disabilities.

Additionally, this auditory environment often drowns out the sounds of human voices. This is detrimental to the child, because human voice has proven integral to the neurodevelopment of preterm newborns. The developmental impacts caused by a lack of early vocal contact is further exacerbated by the absence of interactions between patients/infants and their parents due to professional, personal, and financial burdens, and the inability of the parent to be in the NICU for extended periods of time. Quantitatively, the average time of parental visitation is less than one day each week, leading to fewer opportunities for newborns to hear the voices of their parents. This decrease in engagement has significant consequences for graduates, including impairment of linguistic development and impairment of the child-caregiver relationship during the first twenty-four months of these children's lives.

### SUMMARY

The subject matter described in this document addresses the problem of providing hearing protection for infants while also provided an aural development environment that stimulates the infants' early-stage neurological development.

In general, one innovative aspect of the subject matter described in this specification can be embodied in system including an aural developmental medical device identified by an identifier, the aural developmental medical device comprising: a first audio transducer device and a second audio transducer device that each transduce electrical signals into acoustic sounds, electronics electrically coupled to the first audio transducer device and the second audio transducer device, wherein the electronics are operable to: associate the aural developmental medical device with the identifier, receive, over a communication channel, filtered electrical signals, the filtered electrical signals generated from electrical input signals generated by a microphone device and electronically filtered, the electronic filtering attenuating frequencies above a cutoff frequency that is at least octave above a fundamental frequency of a typical human voice, and provide the filtered electrical signals to the first audio transducer device and the second audio transducer device; wherein the first and second audio transducer devices are operable to be positioned relative to each other so that the first and second audio transducer devices can be respectively positioned over a left ear and a right ear of the patient; and an acoustic processing station comprising one or more processors, one or more memory devices, and one or more communication devices, the acoustic processing station being separate from the aural developmental medical device and operable to: receive the electrical input signals generated by the microphone device, electronically filter the electrical input signals to generate the filtered electrical signals; and send over the communication channel and to the aural developmental medical device, the filtered electrical signals. Other embodiments of this aspect include corresponding methods, apparatus, and computer programs, configured to perform the actions of the methods.

Another innovative aspect of the subject matter described in this specification can be embodied in a method that includes the operations of transmitting, to a processing station, data encoding acoustic sounds detected by a microphone; receiving, at the processing station, the data encoding the acoustic sounds; filters, at the processing station, the data encoding the acoustic sounds to generate filtered data to attenuate frequencies above a frequency f_{c} that is approximately an octave above a fundamental frequency f of a human voice; transmitting, from the processing station to one or more of the aural developmental devices, the filtered data; and presenting the filtered data at the one or more aural developmental devices. Other embodiments of this aspect include corresponding apparatus and computer programs configured to perform the actions of the methods.

Particular embodiments of the subject matter described in this specification can be implemented so as to realize one or more of the following advantages. The systems and methods described herein promote the cognitive development of newborns receiving NICU care by providing audio data for playback to the newborns, while also protecting them from the auditory hazards of their environments. In implementations that do not store the audio data locally, the systems and methods provide an extra layer of privacy protection as at the completion of care, the wearable medical device is disassociated with data stored remotely from the wearable medical device.

The wearable medical device can be inserted into an attachable pouch that is attached to headgear, e.g., a beanie, worn by the infant. In these implementations, the medical device may be easily removed from the pouch when the patient is discharged, thus allowing for easy sanitation and reuse of the wearable medical device.

The use of active filtering enables the pass thorough of human voices while actively suppressing noises outside of a normal human voice range, which, in turn, provides an advantage over passive hearing protection systems that also attenuate human voices. Remote access to a server system in data communication with the wearable medical device allows for family members to upload recordings for periodic playback to the patients, which stimulates neurological development and bonding while the child is receiving treatment in the NICU. Performing active filtering at the station, separate from the devices, reduces power requirements and costs of the individual devices.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an aural development medical device system.

FIG. 2A is an image of an infant wearing an aural development medical device.

FIG. 2B is a block diagram of an example implementation of the aural development medical device.

FIG. 3A is a block diagram of an example acoustic processing station in communication with aural developmental medical devices with integrated microphones.

FIG. 3B is a block diagram of an example acoustic processing station in communication with aural developmental medical devices with associated separate microphone systems.

FIG. 4 is a graph of a frequency response of the sound processing circuit of FIG. 4.

FIG. 5 is a flow diagram of an example process of acoustic processing performed in the system of FIG. 1.

Like reference numbers and designations in the various drawings indicate like elements.

### DETAILED DESCRIPTION

As described above, the NICU environment is often a high-frequency and high-level noise environment. These noises may be unsettling to a newborn, and if loud enough, may also damage a newborn's hearing during prolonged exposure. One solution to protect a child's hearing is to simply provide passive hearing protection for the child, e.g., by use of earmuffs or by use of a sound-insulated incubator. However, this results in aural sensory deprivation, which can hinder the child's very early-stage development.

The technology described in this written description relates to an aural developmental medical device and system. A medical device is worn by an infant while the infant is in a neonatal intensive care unit (NICU). In particular, the device protects the child's hearing while, at the same time, provides aural stimulation for development. An acoustic processing station remote from the wearable medical device manages the acoustic processing for the medical device worn by the infant. In some implementations, a remote server system also enables people associated the child, e.g., parents and siblings, to provide recordings, such as recordings of their voices, for playback to the child.

The technology described in this written description solves the problem of aural sensory deprivation that occurs when protecting an infant's hearing, and thus does not inhibit the development that results from aural stimulation. Additionally, the technology described in this written description provides off-device audio processing, which reduces the on-device processing required for the wearable, which, in turn, also reduces power requirements of the wearable device. This results in a lighter, less cumbersome wearable.

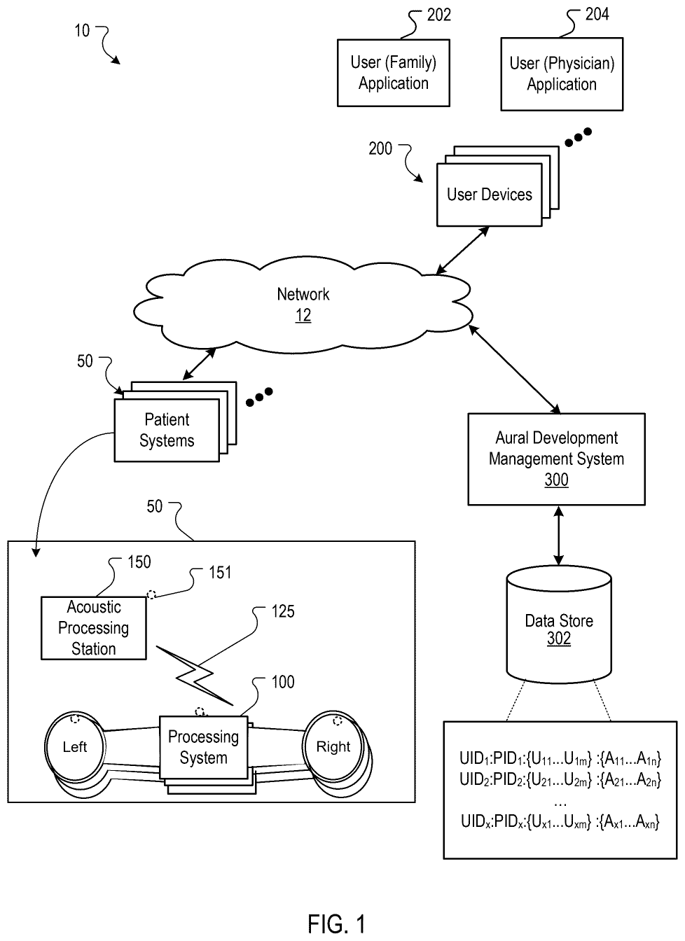

One example implementation as illustrated in FIG. 1, which depicts an aural developmental medical device system 10. The system 10 includes patient systems 50. Each patient system 50 includes one or more aural development medical devices 100 and an acoustic processing station. The system 50 may also include user devices 200, which may operate user (family) applications 202 and user (physician) applications 204. The system 50 may also include an aural development management system 300, which includes a management computer data store 302 that associates recordings with patients.

These features and additional features are described in more detail below.

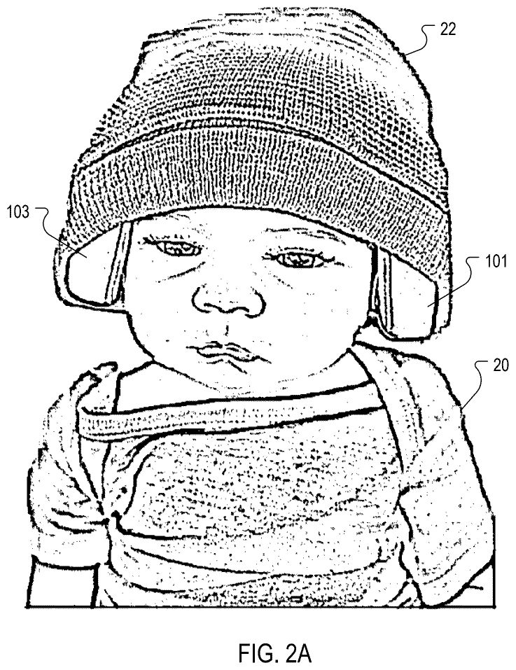

FIG. 2A is an image of an infant 20 wearing an aural development medical device 100. As depicted in FIG. 2, speaker muffs 101 and 103 are worn against the infant's 20 ears. As shown in FIG. 1, the speaker muffs 101 and 103 are supra-aural headphones. In other implementations, circumaural headphones can be used. Other devices that can cover the ears or fit into the ears can also be used.

An example implementation of an aural development medical device 100 is illustrated in FIG. 2B. As will be describe in more detail below, the device 100 is attached to a beanie 22 in a manner that allows the device 100 to be secured in place on the infant's 20 head while in a NICU incubator.

As shown in more detail in FIG. 2B, the device 100 includes speakers 102 and 104. The device 100 optionally includes at least one microphone. The speakers 102 and 104 are first and second audio transducer devices that each transduce electrical signals into acoustic sounds.

In some implementations, a microphone, if included, may be part of the speaker assembly, as shown in phantom by microphone 106 and 108. The microphone is a device that transduces received acoustic sounds into electrical input signals. The microphones may be placed on the outside of the speaker assemblies, and opposite the activation surfaces of the speakers 102 and 104. In another implementation, a single microphone may be used, such as the microphone 110. The placement of the single microphone 110 may be anywhere on the device 100, so long as the placement allows for the microphone 110 to detect sounds within the surrounding environment.

The speaker assemblies 102 and 104 may be enclosed in a padded, hypoallergenic housing to form the speaker muffs 101 and 103. The speaker muffs 101 and 103 offer a level of passive sound attenuation of noises from the surrounding environment that will protect the patient's hearing. In some implementations, the housings may be removed after a patient is discharged, and replaced with new housings after the device 100 is cleaned and sanitized.

The device 100 also includes electronics 120. The electronics 120 are electrically coupled to the first audio transducer device 102, the second audio transducer device 104, and the microphone device. As will be described in more detail below, the electronics 120 include an audio processing subsystem 122. If the device 100 includes a microphone, the audio processing subsystem 122 receives the electrical input signals from the one or more microphone devices and transmits, to the acoustic processing station 150 and over a communication channel, e.g., as indicated by transmission 125 in FIG. 1, data representing the electrical input signals. The processing station 150 filters the electrical input signals to attenuate frequencies above a cutoff frequency f_{c} to produce filtered electrical signals. The filtered electrical signals are sent from the acoustic processing station 150 to the device 100. The device 100 then provides the filtered electrical signals to the first audio transducer device 102 and the second audio transducer device 104.

The processing station 150 can use any appropriate filtering process. For example, the processing station 150 can convert the received digital signals to an analog signal and filter the analog signal using an analog filter, e.g., a single or multi-pole RC filter, and then convert the filtered analog signal back into a digital signal for transmission back to the device 100 for playback. Likewise, the processing station 150 can filter the signals using any appropriate digital filtering process and transmit the filtered digital signal back to the device 100 for playback.

In implementations where two microphones 106 and 108 are used, the filtered electrical signals generated from the processing of the microphone 106 data by the processing station 150 and received from the processing station 150 are provided to the speaker 102, and the filtered electrical signals generated from the processing of the microphone 108 data by the processing station 150 and received from the processing station 150 are provided to the speaker 104. In other implementations that use only one microphone, such as microphone 110, the same filtered electrical signals are provided to both speakers 102 and 104.

The electronics 120 also include a transceiver subsystem 124 that can communicate with other systems wirelessly, such as the acoustic processing station 150. Any appropriate transceiver system can be used, such as one that operates according to personal area network protocol, or one that operates according to a wireless area network protocol, or combinations thereof, or even other protocols.

The electronics 120 can associate the aural developmental medical device 100 with an identifier. For example, the identifier can be the MAC address of a radio device in the transceiver subsystem 124, or can be a unique identifier assigned to the device 100 and stored in a read only memory. Other identifiers, such as serial numbers, etc., can also be used. As will be described in more detail below, the device 100 identifier can optionally be used to associate the device 100 with a unique patent identifier of a patient to which the device 100 is issued. In some implementations, the unique patient identifier can be temporarily stored is a memory of the device 100 while the device is issued to patient identified by the patient identifier, and the patient identifier is used to identify the device. When the patient is discharged, the unique patient identifier is erased from the memory of the device 100.

The transceiver subsystem 124 can transmit and receive data over a network. In some implementations, the transceiver system 124 also receives audio data encoding a recording and provides the data to the CODEC 126 for processing. The audio data is received from the acoustic processing station 150. The processed audio data is then provided to the audio subsystem 122. The audio subsystem 122 then generates, from the audio recording, recorded electrical signals and provides the recorded electrical signals to the first and second audio transducer devices 102 and 104. In this way recordings of family members, such as parents, may be played back to the patient.

Although shown as a component separate from the audio subsystem 122, the CODEC 126 can, of course, be a component of the audio subsystem 122.

In some implementations, the device 100 includes a memory storage that may store the audio data locally on the device 100 for periodic playback. After a patient to which the device 100 has been issued is discharged, the memory storage is overwritten via an automated discharge process. In another implementation, the memory storage may be a small removable storage device, such as a SIMM card, and the device may be removed upon discharge and provided to the parent(s) or caregiver(s) of the patient, or erased, or destroyed.

In other implementations, audio data is not stored on the device, and instead is received through the transceiver system as an audio stream. The CODEC 126 and the audio subsystem 122 then generate, from the audio stream, the recorded electrical signals and provide the recorded electrical signals to the first and second audio transducer devices 102 and 104.

The device 100 includes one or more batteries 132 and 134 to power the electronics 120 and the speakers 102 and 104. Although shown between the speakers 102 and 104 and the electronics 120, the one or more batteries 132 and 134 may be positioned elsewhere on the device 100.

In some implementations, the speakers 102, 104, microphone(s) 106, 108 and 110, electronics 120 and batteries 132 and 134 are contained within, mounted on, or attached to a flexible packaging 140. As shown in FIGS. 1 and 2, the flexible packaging 140 positions the first and second audio transducer devices 102 and 104 relative to each other so that the first and second audio transducer devices 102 and 104 can be respectively positioned over a left ear and a right ear of an infant. In some implementations, the electronics and batteries may be contained with one the speakers, and the other speaker may be connected to the electronics via simple conductors. In this implementation, each speaker may be placed over a respective ear of a patient without the need for the flexible packaging.

Other modules and subsystems may be included in the electronics 120, such as a controller, a memory, LED indicators, etc.

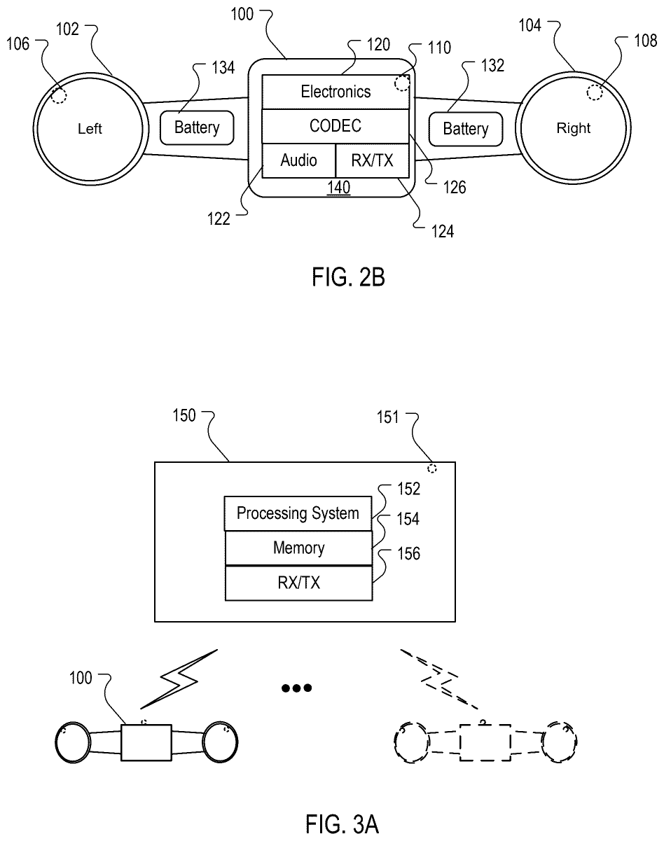

FIG. 3A is a block diagram of an example acoustic processing station 150 in communication with aural developmental medical devices 100 with integrated microphones.

The station 150 includes a processing system 152 of one or more processors, a memory system 154 of one or more memory devices, and a transceiver system 156 of one or more communication devices. The station 150 is operable to receive the electrical input signals generated by the microphone device over a communication channel from an aural developmental medical device, electronically filter the electrical input signals to generate the filtered electrical signals, and send over the communication channel and to the aural developmental medical device, the filtered electrical signals. The station 150 may use any appropriate software or hardware filtering process to filter the acoustic signals.

In some implementations, the station 150 may be paired on a 1:1 basis with a device 100. For example, the station 100 may include the microphone and be mounted within the incubator or nearby the incubator, and the devices 100 may not have microphones. In a variation of this implementation, the devices 100 will have their own respective microphones, and the stations 150 are located separate from the incubators.

Alternatively, the station 150 may be paired on a 1:n basis with n devices, as illustrated by the phantom devices. For example, in the implementation of FIG. 1, each device 100 may have its own microphone, and the station 150 processes all audio data for all the devices 100. In another implementation, the devices 100 may be arranged in “pods” of n devices, e.g., n=2-5, and there is one station for each n device 100. In a variation of this implementation, the devices 100 may not have microphones, and instead a microphone and transmitter may be centrally located within the pod. The station 150 processes the acoustic data transmitted from the microphone transmitter, and then transmits the filtered acoustic data to the devices within the pod. Thus, for a NICU with 25 incubators, with n=5, the NICU may have five pods.

FIG. 3B is a block diagram of an example acoustic processing station 150 in communication with aural developmental medical devices 100 with associated separate microphone systems 160. Each separate microphone system 160 includes a transmitter, and is stationed in an incubator and associated with the aural developmental medical device 100 assigned to the infant in the incubator. The microphone system 160 transmits data encoding acoustic sounds detected by the microphone to the station 150. The data includes the identifier of the microphone system 160. The station 150 generates the filtered data, and, based on the identifier, determines the associated device 100 to which the filtered data is to be addressed, and then transmits the filtered data to the associated device 100.

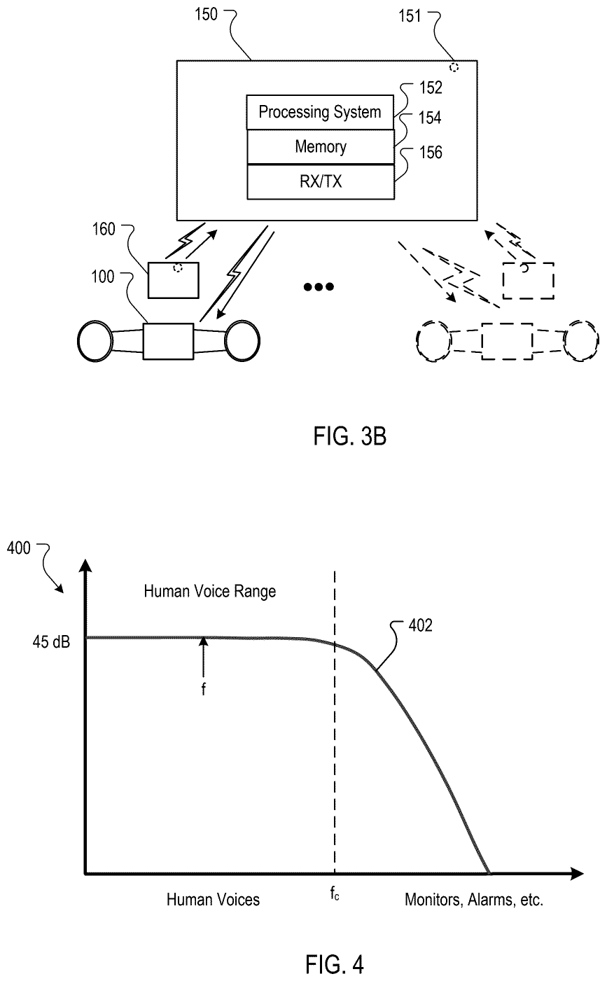

FIG. 4 is a graph 400 of a frequency response 402 of the filtered electronic signals generated by the acoustic processing station 150. The cutoff frequency, in some implementations, is a frequency that is at least an octave above a fundamental frequency f of a typical human voice to generate filtered electrical signals. As shown in FIG. 4, the cutoff frequency f_{c} is approximately an octave above a fundamental frequency f of a human voice. For example, the typical fundamental frequency range of a human female voice is in the rage of 170 Hz to 255 Hz. Thus, in one implementation, the cutoff frequency is approximately 500 Hz. Accordingly, audio frequencies of most human voices are passed, while the audio frequencies of monitors, alarms, and other noises that are typically in excess of this cutoff frequency are attenuated.

The frequency response curve of FIG. 4 is illustrative, and other filter designs with different response curves can also be used. In some implementations, instead of a frequency cutoff, the entire frequency range can be attenuated so that the signal is attenuated to a threshold level, e.g., 45 dB±3 dB.

In some implementations, the audio output level of the speakers 102 and 104 is limited to a sound level that is low enough to ensure that prolonged exposure to the audio will not damage the infants' hearing. In some implementations, this level is 45 dB. Other maximum levels can also be used, however, such as 50 dB or even higher. Likewise, a maximum level can also be less than 45 dB, e.g., 40 dB.

As described above, the station 150 attenuates noises and sounds that are above the normal frequency envelope of the human voice while passing frequencies that are within the normal frequency envelope of the human voice. Moreover, the output level of the speakers is limited so that unfiltered sounds, e.g., the sounds of human voices, detected from the microphone(s) are output at safe levels.

Parents and caregivers of NICU patients are often not able to be with infants during the NICU stay. Accordingly, the aural development management system 10 allows for the parents and caregivers to provide audio recordings of their voices that can be played back to the infants.

As depicted in FIG. 1, the station 150 can also access data recordings for playback on devices 100. In particular, the system 10 may store audio data encoding a recording and play back the recording on the speakers 102 and 104. Again, the audio levels of playback may be limited to the safe audio level. Moreover, during playback, in some implementations, audio detected from the microphone(s) is not processed, and thus the electronics do not provide electrical signal generated in response to an electrical input signal generated by the microphone device during playback. This enables the infant to focus on the sounds of the recordings by reducing distractions that extraneous voices and noises might otherwise cause.

Returning to FIG. 1, the system 10 includes patient systems 50, such as the device 100 of FIG. 2B and the station 150 of FIG. 3. The stations 150 are in data communication over the network 12 with an aural development management system 300. The network 12 may be a computer, such as a local area network (LAN), wide area network (WAN), the Internet, or a combination thereof. More generally, any protocol that is appropriate for transmitting recorded audio can be used in the network 12.

The aural development management system 300 may be realized by one or more computers in data communication with each other and running an application(s) that perform the operations described below, or programmed to perform the operations described below.

The system also includes one or more applications on user devices 200, such as user (family) applications 202 and user (physician) applications 204 that each run on a user device 200. The user devices 200 may be, for example, smart phones or computers. Through a family application 202, a user may record his or her voice and upload the recording to the system 300 for later playback to a particular patient device 100. The family application 202 may also allow for the deletion of certain recordings, and for the scheduling of playback of the recordings according to a playback schedule.

The physician application 204 may include the same functionalities of the family application 202, and may also include other functionalities that are reserved for physicians, physician assistants, nurses, and other hospital staff. These functionalities may include overriding or adjusting playback schedules set by users of the family application 202, setting cumulative playback time for a time period, and associating and de-associating a particular device 100 with a particular patient.

Additionally, the station 150 may store recordings that are broadcast to multiple devices 100 simultaneously. For example, the station 150 may broadcast a recording of the ambient sounds as heard from a mother's womb during the later stages of pregnancy, as these sounds are calming to newborns. Examples of such sounds include a human heartbeat; the sound of breathing; other ambient sounds of the human body; and combinations thereof. The station 150 may also broadcast a recording of a soothing voice, e.g., as recorded by a voice talent.

Each device 100 is issued to a patient and has a corresponding unique identifier UID. The device may be a MAC address, or an address stored in a memory on the device 100, such as a patient identifier, or some other unique identifier. Upon issuance, the unique identifier UID is associated with a patient identifier PID. The PID may be a hospital patient identifier for the patient, and, as previously mentioned, may be the same as the UID.

In some implementations, each UID is a permanent identifier assigned to a particular user device. In other implementations, the UID may be temporarily stored on the device 100 in a memory, and may, for example, be the patient identifier PID. Each particular device 100 may be accessed based on the UID of the device 100.

For each patient, one or more user identifiers U are associated with the patient identifier of the patient. This may be done by setting up an account through the physician application 204, or by setting up the account after downloading and launching a family application to a user device 200. For example, once a device 100 with a user identifier UID is issued to a patient with a patient identifier PID, the UID and PID are associated with each other. Thereafter, a unique uniform resource identifier (URI) link may be generated and sent to a family member listed as a caregiver of the patient. The family member may download the family application 202 by selecting the link on the user device 200, and then proceed to set up an account on the aural development management system 300. The account is linked to corresponding identifiers. During this time, the family member may add additional users to the account, should the family member choose to do so.

The users that are associated with a patient identifier in an account are authorized to provide audio data encoding recordings for presentation on the aural developmental medical device 100 issued to the patient. This can be done, for example, by a particular user recording the audio files by use of the family application 202. Once recorded, the audio file is transmitted from the user device 202 to the system 300 via the network 12. Based on the particular user as identified by the user identifier, the system 300 determines the particular patient associated with the particular user. After the particular patient is determined, the system 300 stores in a data store 302, in association with the particular patient, the audio data encoding the recording and received from the user device.

Thus, for each particular device 100, the following data set is established:
UIDₓ: PIDₓ: {Uₓ₁ . . . . Uₓₘ}: {Aₓ₁ . . . . Aₓₙ}
where:
UIDₓ is the unique identifier of the device 100;
PIDₓ is the patient identifier of the particular patient;
{Uₓ₁ . . . . Uₓₘ} are the users associated with the particular patient identifier; and
{Aₓ₁ . . . . Aₓₙ} are the audio files recorded by the users associated with the particular patient identifier.

The audio files are stored in the data store 302 and are addressed by and accessible by use of the URI provided by the system 300 to the corresponding user devices 100. In some implementations, the audio files may be of any playback length, and need not be of a fixed duration or size.

In response to a request from an acoustic processing station 150, the system 300 sends, to the acoustic processing station 150 issued to the particular patient, the audio data encoding the recordings. The data may also include a playback schedule, the patient system 50 then processes the audio files to generate recorded electrical signals and provide the recorded electrical signals to the first and second audio transducer devices according to the playback schedule.

The audio files typically record human voices, such as the voices of the mother and other family members. Other recordings can also be recordings of an internal sound of a human body, such as a recording of the sounds of the mother's body. In some implementations, the recordings of the sounds of the mother's body may be played back constantly between the playback of other recordings. Given that each patient is recently born, the familiar internal sounds of the mother's body will tend to calm and comfort the patient. In variations of this implementation, the body sounds may be overlaid with the mother's voice, or recorded while the mother is speaking.

In some implementations, upon recording on a user device 200, or when being stored by the system 300, the audio data is filtered to attenuate frequencies that are above the cutoff frequency f_{c}. Accordingly, when the audio is processed by the station 150, filtering of the audio need not be performed on the device station 150.

In some implementations, the microphone devices need not be mounted on the device 100 itself, and instead are placed within the incubator. The microphone device may be connected to a transmitter/receiver that is associated with the device 100 assigned to the infant. For example, at a user interface, a patient identifier may be associated with the device 100 and the separate microphone device, the latter two of which are identified by unique identifier, e.g., MAC addresses or network addresses. In this way, the sounds detected by the microphone within the infant's incubator are transmitted to the processing station 150, processed and filtered, and transmitted back to the device 100 for the infant to hear.

In some implementations, the processing station 150 may include a microphone 151. Sounds detected by the microphone 151 may be filtered and transmitted to one or more devices 100. For example, the processing station 150 may be located at a nurses' station, and a nurse on duty may periodically activate the microphone and say something intended to be soothing to the infants. In variations of this implementation, the nurse may select one or more devices with which to transmit simultaneously. The available devices may be displayed in a menu, and the nurse may select which devices the procession station 150 is to transmit to. In this way, the nurse may speak to one, several or all of the infants in the NICU at any one time.

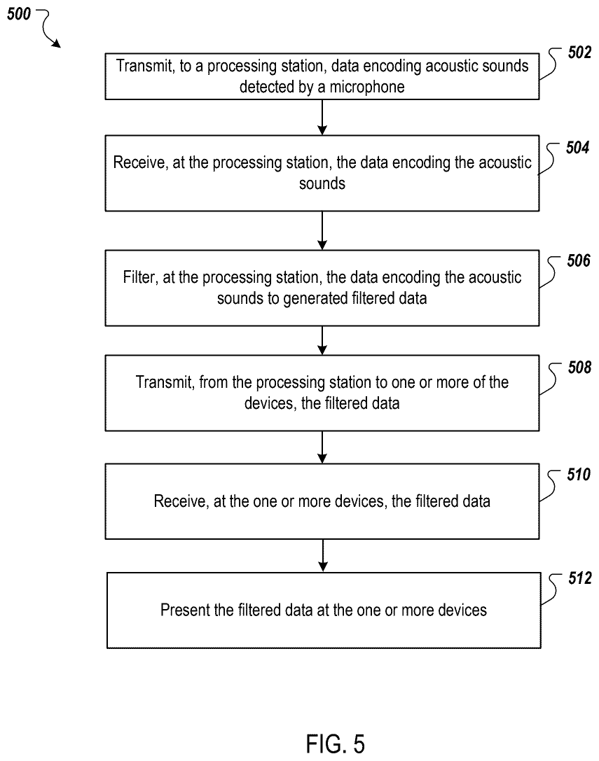

FIG. 5 is a flow diagram of an example process 500 of acoustic processing performed in the system of FIG. 1. The process 500 can be performed by the computing devices of FIGS. 1, 2B and 3, which are described above an in more detail below.

The process 500 transmits, to a processing station, data encoding acoustic sounds detected by a microphone (502). The microphone can be a microphone located on a wearable developmental device 100, or a microphone located within an incubator of a patient, or a microphone located remotely from the incubator and device, such as a nurses' station.

The process 500 receives, at the processing station, the data encoding the acoustic sounds (504). For example, as described above, the data may be generated from sounds detected by one or more microphones on the device 100. Alternatively, the data may be from sounds from a microphone system that is separate from the device 100 and mounted in an incubator and associated with the device 100, which, in turn, is associated with a patient. In these two cases, the data may also have identifier data that identifies the device 100 with which the data is associated. The identification can be by a patient identifier, a network address, or any other appropriate identification scheme. In yet another implementation, the sounds may be received from a microphone at a nurses' station, separate from the device 100. In this case, the data may include one or more identifiers that are associated with one or more devices 100 to which the later filtered sound is to be transmitted.

The process 500 filters, at the processing station, the data encoding the acoustic sounds to generate filtered data (506). For example, the process can filter the acoustic sounds by means of either an analog filter or digital filter, and appropriate processing, to attenuate frequencies above a frequency f_{c} that is approximately an octave above a fundamental frequency f of a human voice. For example, the typical fundamental frequency range of a human female voice is in the rage of 170 Hz to 255 Hz. Thus, in one implementation, the cutoff frequency is approximately 500 Hz.

The process 500 transmits, from the processing station to one or more of the devices, the filtered data (508). As described above, if the filtered data are to be broadcast to all devices 100, then the processing station 150 broadcasts the filtered to every device 100. Alternatively, if filtered data are to be transmitted to only one device, or fewer than all devices, then the filtered data is associated with the identifier of the devices and the devices to which the filtered data transmit are received by the devices.

The process 500 receives, at the device, the filtered data (510). As described above, the filtered data can be received by one, several or all devices, depending on whether the filtered data are transmitted to one or several devices, or broadcast to all devices.

The process 500 presents the filtered data at the device (512). As described above, the filtered data is processed by the device 100 electronics and the filtered sounds are generated by the speakers of the device.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., a FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback;

and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a user computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include users and servers. A user and server are generally remote from each other and typically interact through a communication network. The relationship of user and server arises by virtue of computer programs running on the respective computers and having a user-server relationship to each other. In some embodiments, a server transmits data (e.g., an HTML page) to a user device (e.g., for purposes of displaying data to and receiving user input from a user interacting with the user device). Data generated at the user device (e.g., a result of the user interaction) can be received from the user device at the server.

While this specification contains many specific implementation details, these should not be construed as limitations on the scope of any features or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A system, comprising:
an aural developmental medical device identified by an identifier, the aural developmental medical device comprising:
a first audio transducer device and a second audio transducer device that each transduce electrical signals into acoustic sounds;
electronics electrically coupled to the first audio transducer device and the second audio transducer device, wherein the electronics are operable to:
associate the aural developmental medical device with the identifier;
receive, over a communication channel, filtered electrical signals, the filtered electrical signals generated from electrical input signals generated by a microphone device and electronically filtered, the electronic filtering attenuating frequencies above a cutoff frequency that is at least octave above a fundamental frequency of a typical human voice; and
provide the filtered electrical signals to the first audio transducer device and the second audio transducer device;
wherein the first and second audio transducer devices are operable to be positioned relative to each other so that the first and second audio transducer devices can be respectively positioned over a left ear and a right ear of the patient; and
an acoustic processing station comprising one or more processors, one or more memory devices, and one or more communication devices, the acoustic processing station being separate from the aural developmental medical device and operable to:
receive the electrical input signals generated by the microphone device;
electronically filter the electrical input signals to generate the filtered electrical signals; and
send over the communication channel and to the aural developmental medical device, the filtered electrical signals.

2. The system of claim 1, wherein:
the aural developmental medical device further comprises the microphone, wherein the microphone transduces received acoustic sounds into the electrical input signals, and the electronics are further operable to send, to the acoustic processing station and over the communication channel, the electrical input signals; and
the acoustic processing station receives, over the communication channel, the electrical input signals generated by the microphone device.

3. The system of claim 1, wherein the acoustic processing station further comprises the microphone, wherein the microphone transduces received acoustic sounds into the electrical input signals.

4. The system of claim 1, wherein:
the acoustic processing station is further operable to:
receive data over one or more networks and communicate data over the one or more networks, wherein the communication channel over which the electronics of the aural development medical device is included in the one or more networks;
access, over the one or more networks, audio data encoding a recording, the audio data generated by a user authorized to provide audio data for presentation on the aural developmental medical device issued to the patient, the audio data stored at a location remote from the aural developmental medical device;
receive, over the one or more networks, the audio data; and
send, to the aural developmental medical device, the audio data; and
the aural developmental medical device is further operable to:
receive, over the one or more networks, the audio data; and
generate, from audio data encoding the recording, the recorded electrical signals and provide the recorded electrical signals to the first audio transducer device and the second audio transducer device for playback of the audio data.

5. The system of claim 4, further comprising and aural development management computer separate from the acoustic processing station and separate from the aural developmental medical device, comprising:
one or more computer devices; and
instructions stored in a non-transitory computer readable medium that are executable by the one or more computer devices, and upon such execution cause the one or more computer devices to perform operations comprising:
store an association of patients and users, wherein the association establishes, for each patient:
an aural developmental medical device issued to the patient and identified by a unique identifier;
one or more users that are authorized to provide audio data for presentation on the aural developmental medical device issued to the patient;
receive, over a computer network and from a user device that is remote from the one or more computer devices and associated with a particular user, audio data encoding a recording;
determine, based on the particular user, the particular patient associated with the particular user;
store, in the association for the particular patient, the audio data encoding the recording and received from the user device; and
send, to the acoustic processing station, the audio data encoding the recording associated with the identifier of the aural developmental medical device issued to the particular patient.

6. The system of claim 4, wherein the system comprises:
a plurality of the aural developmental medical devices, wherein the identifier of each aural developmental medical device is an identifier unique to that aural developmental medical device and is associated with a particular patient; and
the acoustic processing station is further operable to:
access, over the one or more networks, for each of the aural developmental medical devices, audio data encoding a recording, the audio data generated by a user authorized to provide audio data for presentation on the aural developmental medical device issued to the patient based on the unique identifier;
receive, over the one or more networks, the audio data; and
send, to the aural developmental medical device based on the unique identifier, the audio data.

7. The system of claim 4, wherein the acoustic processing station is further operable to periodically send, to the aural developmental medical device, the audio data according to a playback schedule.

8. The system of claim 1, wherein the cutoff frequency is approximately 500 Hz.

9. A computer-implemented method, comprising:
transmitting, to a processing station, data encoding acoustic sounds detected by a microphone;
receiving, at the processing station, the data encoding the acoustic sounds;
filters, at the processing station, the data encoding the acoustic sounds to generate filtered data to attenuate frequencies above a frequency f_{c} that is approximately an octave above a fundamental frequency f of a human voice;
transmitting, from the processing station to one or more of the aural developmental devices, the filtered data; and
presenting the filtered data at the one or more aural developmental devices.

10. The computer-implemented method of claim 9, wherein the data encoding the acoustics sounds is generated from acoustic sounds detected by a microphone on an aural developmental device.

11. The computer-implemented method of claim 10, wherein transmitting, from the processing station to one or more of the aural developmental devices, the filtered data comprises transmitting, from the processing station to only the aural developmental device from which the data encoding the acoustics sounds was received.

12. The computer-implemented method of claim 9, wherein the data encoding the acoustics sounds is generated from acoustic sounds detected by a microphone system separate from and associated with aural developmental device.

13. The computer-implemented method of claim 12, wherein transmitting, from the processing station to one or more of the aural developmental devices, the filtered data comprises transmitting, from the processing station to only the aural developmental device associated with the microphone system that is associated with aural developmental device.

14. The computer-implemented method of claim 12, wherein transmitting, from the processing station to one or more of the aural developmental devices, the filtered data, comprises transmitting the filtered data to a plurality of aural developmental devices.
